(19) United States
(12) Patent Application Publication
(10) Pub. No.: US 2024/0237896 A1
(43) Pub. Date: Jul. 18, 2024

(54) OPHTHALMOSCOPES

(71) Applicant: EPIPOLE LTD, Dunfermline, Fife (GB)

(72) Inventors: Craig ROBERTSON, Dunfermline (GB); Derek RODGER, Dunfermline (GB); Charles WARDROP, Dunfermline (GB); Ian MURRAY, Dunfermline (GB); Philip BENSON, Dunfermline (GB); Robert HENDERSON, Dunfermline (GB)

(21) Appl. No.: 18/563,266

(22) PCT Filed: May 27, 2022
(86) PCT No.: PCT/GB2022/051369

§ 371 Date: Nov. 21, 2023

(30) Foreign Application Priority Data
May 28, 2021 (GB) 2107697.1

(51) Int. Cl. A61B 3/14; A61B 3/00; A61B 3/12; A61B 3/15

(52) U.S. Cl. CPC A61B 3/145; A61B 3/0025; A61B 3/005; A61B 3/1208; A61B 3/152; A61B 2560/0425

(57) ABSTRACT

The invention relates to an ophthalmoscope (10) configured to acquire video of an eye. The ophthalmoscope (10) comprises a light source, an imaging apparatus (20), and a processor. The light source emits a beam of non-coherent light which in use impinges on the eye. The imaging apparatus (20) acquires plural images of at least part of the eye, each of the plural images acquired when non-coherent light from the light source impinges on the eye. The processor is configured to at least one of: control the imaging apparatus (20) such that time adjacent images are acquired with a predetermined period therebetween; and record a time of acquisition of each of a plurality of the plural images and to associate each time of acquisition with the respective image. The acquired plural images are thus related in time to one another and thereby constitute video of the eye.

### FIELD OF THE INVENTION

The present invention relates to ophthalmoscopes and more specifically an ophthalmoscope which emits a beam of non-coherent light that impinges on the eye during use of the ophthalmoscope. The present invention also relates to a method of operation of an ophthalmoscope comprising emitting a beam of non-coherent light from the ophthalmoscope that impinges on the eye during use of the ophthalmoscope.

### BACKGROUND ART

Ophthalmoscopy is performed for medical screening programmes, medical diagnosis, and monitoring disease progression. It is known in ophthalmoscopy to use electronic camera-based technology to acquire an image of the eye. For example apparatus comprising a retina camera and image acquisition circuitry is used in fundus photography by medical clinicians to acquire an image of the retina. Acquired retinal images are normally analysed later by a specialist to detect and evaluate symptoms of eye disease such as glaucoma or age-related macular degeneration.

It is further known to acquire a few images of a subject's eye by way of a camera and image acquisition circuitry. Although such images are acquired in order there is a lack of timing information associated with each image whereby such images do not constitute video when taken together because of the inability to place each of the images in its proper location on the time axis.

Known electronic camera-based ophthalmology apparatus typically comprises a scanning laser which causes laser light to impinge on the eye during image acquisition. Although a scanning laser is suitable for acquisition of a single image or perhaps two or three images subject to their being infrequently acquired, there is risk of injury to the eye if laser light impinges on the eye for long enough to acquire video of the eye. There is therefore a prejudice against acquiring video of the eye.

The present inventors have become appreciative of the medical value in performing video ophthalmology. The present invention has been devised in light of the inventors' appreciation and against a background of unsuitability of typical electronic camera-based ophthalmology apparatus for acquisition of video and a consequential prejudice against acquisition of video. It is therefore an object for the present invention to provide an improved ophthalmoscope configured to acquire video of the eye of a human or animal subject. It is a further object for the present invention to provide an improved method of performing ophthalmology with an ophthalmoscope configured to acquire video of the eye of a human or animal subject.

### STATEMENT OF INVENTION

According to a first aspect of the present invention there is provided an ophthalmoscope configured to acquire video of an eye, the ophthalmoscope comprising:
a light source emitting a beam of non-coherent light which in use impinges on the eye;
an imaging apparatus which acquires plural images of at least part of the eye, each of the plural images acquired when non-coherent light from the light source impinges on the eye; and
a processor configured to at least one of: control the imaging apparatus such that time adjacent images are acquired with a predetermined period therebetween; and to record a time of acquisition of each of a plurality of the plural images and to associate each time of acquisition with the respective image, whereby the acquired plural images are related in time to one another and thereby constitute video of the eye.

The ophthalmoscope comprises a light source emitting a beam of non-coherent light which in use impinges on the eye. In contrast to laser light, non-coherent light may impinge on the eye with reduced risk of damage being caused to the eye and in particular when the light impinges on the eye for long enough to acquire video of the eye. The ophthalmoscope may be operative such that light impinges continuously on the eye during acquisition of video of the eye, for example, by the light source emitting the beam of non-coherent light continuously during acquisition of the video.

The ophthalmoscope further comprises an imaging apparatus which acquires plural images of the eye. Each of the plural images is acquired when non-coherent light from the light source impinges on the eye. The ophthalmoscope also comprises a processor.

The processor is configured to at least one of: control the imaging apparatus such that time adjacent images are acquired with a predetermined period therebetween; and to record a time of acquisition of a plurality of the plural images and to associate each time of acquisition with the respective image, whereby the acquired plural images are related in time to one another and thereby constitute video of the eye. Where less than all of the acquired images have a recorded time of acquisition, the remaining acquired images are time related by way of a predetermined period between time adjacent images. For example, each of the plural images may be spaced by a predetermined period from a neighbouring image whereby each of the plural images can be placed in its proper location on the time axis relative to the other images. By way of another example, each of the plural images may be associated with a time of acquisition, such as by way of storage together in a data store or linked by way of a data structure, whereby each of the plural images can be placed in its proper location on the time axis relative to the other images. The approaches of spacing an image by a predetermined period from a neighbouring image and associating a time of acquisition with each of a plurality of the plural images, i.e. each of at least two of the plural images, may be applied in respect of the same plurality of acquired images. For example, nineteen of twenty images may be subject to spacing by a predetermined period, one of these nineteen images may be associated with a first acquisition time, and the twentieth image may be associated with a second acquisition time whereby each of all twenty images can be placed in its proper location on the time axis relative to the other images. Irrespective of which of the approaches or combination thereof is taken, the acquired plural images constitute video of the eye.

As mentioned above, the present inventors have become appreciative of the medical value in performing video ophthalmology.

Some eye pathologies are three dimensional involving changes across the retina surface and also changes in the profile of the retina, i.e. in a direction orthogonal to the retina surface. By way of a first example, age-related macular degeneration involves the macula lifting up due to fluid beneath. By way of a second example, glaucoma may involve the rim around the optic nerve head becoming puckered. By way of a third example, retinoblastoma in children may involve “fingers” of pathology protruding from the surface of the retina. As discussed above, known ophthalmoscopes which typically comprise a scanning laser are capable of acquiring a few images. However, a few images are usually insufficient for investigation of three-dimensional pathologies. Three-dimensional pathologies become more evident from video of the retina which is acquired as the direction and/or position of the ophthalmoscope is changed relative to the eye whereby an acquired part of the retina in the video changes over time. The user of the present ophthalmoscope may, for example, change the direction and/or position of the ophthalmoscope whereby the ophthalmoscope scans across the retina surface or part thereof. Three dimensional features and surface texture may thus become very evident. As described further below, constituting the ophthalmoscope as a hand-held ophthalmoscope may provide for ease of change in direction and/or position of the ophthalmoscope.

As discussed above, known ophthalmoscopes which typically comprise a scanning laser are capable of acquiring a few images. However, a few images are usually insufficient for investigation of biological processes. On the other hand, biological processes may become evident with video. Where the present ophthalmoscope acquires video of a same part of the eye and more specifically the same part of the 10 retina, blood transport in the form of pulsing and distortion of blood vessels may be evident from the acquired video. Change in deformation of the optic nerve head and microscopic movements in nerve tissue may also be evident from the acquired video. Evidence of such biological processes may be of considerable value in medical screening, medical diagnosis, and disease progression monitoring. For example, monitoring movement of blood through blood vessels in the eye has allowed researchers to estimate not only heart rate but also the pressure inside the eye. The carotid artery splits into two parts, with the first part reaching the brain and the second part reaching the eyes. If the pressure in the second part of the carotid artery is known, the pressure in the first part of the carotid artery can be inferred. The correspondence between pressures in the first and second parts of the carotid artery has allowed researchers to estimate the intra-cranial pressure (ICP) with some accuracy. Since high ICP can be fatal in the case of head injuries, malaria, and other diseases this is a potentially life-saving application of an ophthalmoscope according to the present invention.

Conversely low blood pressure is known to be associated with early onset neurodegenerative diseases like Parkinson's and Alzheimer's. An ophthalmoscope according to the present invention may therefore also provide for detection of such neurodegenerative diseases.

Monitoring of biological processes may benefit from the ophthalmoscope acquiring video of the same part of the retina. Where the direction of the ophthalmoscope is liable to change during video acquisition, such as when the present ophthalmoscope is constituted as a hand-held ophthalmoscope, the effect of change in direction of the ophthalmoscope may be addressed by video stabilisation. The ophthalmoscope may therefore be configured to carry out image stabilisation and more specifically digital image stabilisation.

Image stabilisation may comprise determining a registration and more specifically a rigid registration, which may involve translation and rotation, between acquired images. Determining the rigid registration may comprise determining an x, y translation between two acquired images. The x, y translation may be determined by applying Fast Fourier Transform (FFT) matching and Normalised Cross Correlation (NCC). The x, y translation may be further determined by FFT deconvolution and finding a peak in the inverse shifted FFT result.

As discussed above, constituting the ophthalmoscope for hand-held use may be advantageous. The ophthalmoscope may therefore be a hand-held ophthalmoscope and more specifically an ophthalmoscope which is holdable in one hand. The hand-held ophthalmoscope may comprise a main body which sized to be hand-held and more specifically sized to be held in one hand. The main body may be shaped such that it may be gripped in one hand.

The main body may define at least one internal space in which the light source, the imaging arrangement, and the processor are accommodated. The main body may define a window through which the non-coherent light from the light source is directed towards the eye. The imaging arrangement may acquire the plural images by way of an imaging path which passes through the window.

The advantage of a hand-held ophthalmoscope is discussed above. The present inventors have appreciated that it is easier to align a hand-held ophthalmoscope with a patient if video is acquired. In known desktop ophthalmoscopes movement of the subject is restricted by use of a chin rest. Use of a chin rest leaves only two degrees of rotational freedom of the eye to contend with for alignment. Use of a hand-held ophthalmoscope, on the other hand, results in fourteen degrees of freedom, i.e. six degrees of freedom in the hand position of the clinician holding the hand-held ophthalmoscope, six degrees of freedom in the position of the subject, and two degrees of rotational freedom of the eye. The fourteen degrees of freedom may make it difficult for a clinician to align a hand-held ophthalmoscope with a patient. However, if the hand-held ophthalmoscope acquires video of the patient's eye, the hand-held ophthalmoscope may be aligned much more readily. Ease of alignment may provide for acquisition of a single image of interest. A single image of a particular part of the retina may contain vital data in certain diagnostic applications. Even if the clinician is not seeking alignment for acquisition of the single image of interest, plural images constituting the acquired video will most likely comprise the image of interest if the clinician acquires a generally appropriately directed video of sufficient duration.

Pathologies may be searched for by way of video. An image acquired by an ophthalmoscope is usually of less than all of the retina. An ophthalmoscope which acquires video according to the present invention, and more particularly a hand-held ophthalmoscope, may have its orientation changed relative to the eye whereby all of the retina may be examined. Pathologies in the periphery may thus be searched for and recorded. Recording with video may be advantageous because video imparts a sense of journey to the clinician with the sense of journey providing for ease of recall by the clinician of location and nature of pathologies.

Frequency of acquisition of images by the present ophthalmoscope depends on the application to hand. It has been found advantageous in investigation of the biological processes described above to acquire images at a rate of between 10 and 50 frames per second. This range of acquisition rate may accommodate a relatively wide range of heart rates from 50 beats per minute in adults to 120 beats per minutes in children. Biological processes may be investigated to sufficient extent and depending on circumstances with a range of rate of acquisition of images which is narrower at at least 15 frames per second and/or no more than 25 frames per second. The ophthalmoscope may be configured accordingly. More specifically, the processor may be configured to control the rate of acquisition of images, for example, by controlling the imaging apparatus.

The ophthalmoscope may acquire lossless video. Lossy video, such as of jpg format, tends to cause distortion and ringing. Lossless video, on the other hand, is more advantageous in being less prone to distortion or ringing.

It is known to use infrared light as a guide beam for aligning an ophthalmoscope with the eye. Infrared light is used because it does not cause the pupil to contract whereby the aperture presented by the pupil for imaging of the retina is not reduced. However, the blink response is also suppressed because infrared light is invisible to the eye. Prolonged use of infrared light is therefore liable to heat up the eye. The present inventors have discovered that use of red light may address this disadvantage. Red light causes little pupil contraction while not suppressing the blink response because of the visibility of red light. Use of red light therefore mitigates eye heating by providing for continued blinking. Furthermore, and where the eye is at least partially dark adapted before use of the red light, the eye returns quickly to its dilated state upon removal of the red light. Ophthalmology is usually performed on at least partially dark adapted eyes whereby this latter benefit may be realised in acquisition of video with the present ophthalmoscope upon alignment of the ophthalmoscope by way of the red light.

The light source may therefore emit a beam of non-coherent red light. Furthermore, the ophthalmoscope may comprise a display which is viewable by a user of the ophthalmoscope. Where the ophthalmoscope comprises a main body, the display may be mounted on the main body. The ophthalmoscope may be configured, such as under control of the processor, to display on the display images acquired by the imaging apparatus. The ophthalmoscope may be operative, such as under control of the processor, such that red light from the light source is emitted from the ophthalmoscope while images are acquired and the display displays the acquired images. The emitted red light may therefore be used as a guide beam for alignment of the ophthalmoscope with the eye while feedback on alignment is provided to the user by way of the display.

The wavelength of the emitted red light may lie substantially in the range of 620 nm to 740 nm. The wavelength of infrared light has an accepted upper limit of 1 mm. The lower limit for the wavelength of infrared light depends on the division scheme with the lower limit being 750 nm according to the commonly used sub-division scheme, 780 nm according to the ISO 20473 scheme, and 700 nm according to the CIE division scheme. Red light of longer wavelength may provide for less pupil contraction while suppressing the blink response to greater extent whereas red light of shorter wavelength may provide for more pupil contraction while suppressing the blink response to less extent. There may therefore be a balance to be struck between extent of pupil contraction and extent of suppression of the blink response.

The present inventors have discovered emitted red light of wavelength lying substantially in a range which is narrower at no more than 700 nm to provide improved performance in respect of compromise between pupil contraction and blink suppression. More optimal performance in respect of this compromise has been found when the wavelength of the emitted red light lies substantially in a range which is narrower at at least 625 nm and/or no more than 680 nm, and more specifically at least 635 nm and/or no more than 660 nm, with 650 nm preferred in certain circumstances. The light source may therefore emit a beam of non-coherent red light of wavelength lying substantially in one of these ranges or having a wavelength of 650 nm.

Alternatively or in addition to use of red light as a guide beam, red light may be used to advantage in image acquisition. Red light may be effective in imaging the choroid (i.e. the very back of the retinal substrate), which can show indicative ischemia (i.e. darkening) when adverse pathologies are present in other layers of the retina. The ophthalmoscope may be operative, such as under control of the processor, to acquire a single image or video when red light impinges on the eye. The ophthalmoscope may be so operative for diagnostic purposes rather than alignment by use of a light source emitting a beam of red light.

Light of different wavelength compositions may be useful for different diagnostic purposes.

Amber light may be used to image vessel structures and pathological artifacts, such as microaneurysms, while having to some extent the above-described characteristics of red light, such as limited pupil contraction. Amber light is absorbed closer to the front of the retinal surface and is therefore better than red light for diagnostic imaging of certain vessel structures and pathological artifacts. The light source may therefore emit a beam of non-coherent amber light. The wavelength of the light may lie substantially in the range of 585 nm to 625 nm. The present inventors have found light of wavelength lying in a range which is narrower at at least 585 nm and no more than 600 nm, and more specifically at least 585 nm and no more than 595 nm, to be of more optimal effect with 589 nm preferred in certain circumstances.

Broad spectrum white light may capture most of the layers at the front of the retina, i.e. the interior side of the retina, in a single image. However, the light all arrives at the imaging apparatus at the same time which means that all of the structural and colour information are acquired simultaneously. Simultaneous acquisition of information may be good for some pathologies but less so for others. The light source may therefore emit a beam of substantially white light. The spectrum of the white light may extend between 400 nm and 740 nm and more specifically in a narrower range, which is more optimal depending on diagnostic circumstances and which is at least 450 nm and/or no more than 690 nm.

In view of light of different wavelength composition being useful for different purposes, i.e. alignment and diagnosis, or different diagnoses, the present inventors have devised an ophthalmoscope capable of emitting light of different wavelength compositions and more specifically light of different wavelength compositions at different times. The ophthalmoscope may be configured accordingly. More specifically, the ophthalmoscope may comprise plural light sources which each emit light of a different wavelength composition. For example, a first light source may emit red light for alignment and/or diagnosis and a second light source may emit white light at a different time for diagnosis. The ophthalmoscope may be operative, such as under control of the processor in response to user interaction with the ophthalmoscope, for example by way of user controls comprised in the ophthalmoscope, to select one of the plural light sources for emission of light and/or to change between the plural light sources.

The ophthalmoscope may be optically configured such that the paths followed away from the light sources by the plural lights of different wavelength composition are at least overlapping along a part of their length. Overlapping of the paths may be at and adjacent an objective end of the ophthalmoscope. Where the ophthalmoscope comprises the main body and window described above, overlapping of the paths may be at and adjacent the window.

In light of appreciation of the benefits of use of light of different wavelength compositions, the present inventors have configured the ophthalmoscope to emit a first number of lights of different wavelength composition when the ophthalmoscope comprises a second number of light sources, the first number being greater than the second number. More specifically, the ophthalmoscope may be optically configured to at least one of: combine light emitted by at least two light sources; and filter light emitted by at least one light source to thereby change the wavelength composition of the light emitted by the at least one light source.

The ophthalmoscope may comprise a first light source emitting white light and at least one further light source each emitting light of a different colour. For example, the ophthalmoscope may comprise a second light source emitting red light and a third light source emitting amber light. In addition, the ophthalmoscope may comprise at least one filter which filters light of substantially the same wavelength(s) as wavelength(s) of light emitted by the least one further light source, the least one filter disposed in a path of light emitted by the first light source. Furthermore, the ophthalmoscope may be optically configured to combine light emitted by the least one further light source with the light emitted by the first light source after filtering by the at least one filter. The first and at least one further light source may be operative to emit light whereby white light is emitted from the ophthalmoscope. In this mode of operation the at least one further light source may reintroduce light of at least one colour which has been filtered by the at least one filter. In another mode of operation the first light source is operative to emit white light and the at least one further light source is inoperative whereby there is no reintroduction of light after filtering. An ophthalmoscope structured and operative in this fashion may thus provide for greater scope of illumination of the eye by different colours. For example, indocyanine green or fluorescein angiography imaging may be provided.

The ophthalmoscope may comprise a first filter disposed relative to the first light source, i.e. the white light emitting light source, such that the first filter receives light emitted by the first light source, the first filter filtering light of a colour corresponding to the colour of light emitted by a second light source, for example red light, from the light received from the first light source. The first filter may be dichroic. The first filter may reflect to thereby filter light of the colour corresponding to the colour of light emitted by the second light source. Otherwise the first filter may transmit light emitted by the first light source which is of shorter wavelength than the reflected light. Alternatively or in addition, the first filter may be a shortpass filter, light of the colour corresponding to the colour of light emitted by the second light source being reflected because it falls outside the passband of the shortpass filter.

Light emitted by the first light source may impinge on a first side of the first filter whereby light of the colour corresponding to the colour of light emitted by the second light source is filtered, and more specifically is reflected, by the first filter whereas light otherwise is transmitted by the first filter. More specifically, the first filter may be oblique, such as at 45 degrees, to the path of the white light emitted by the first light source.

Light emitted by the second light source may impinge on a second, opposite side of the first filter. The light from the second light source may be reflected by the first filter whereupon the reflected light is combined with the light emitted from the first light source and transmitted by the first filter to thereby in effect reintroduce light filtered by the first filter. The first filter may be oblique to, such as at 45 degrees to, the path of the light received from the second light source.

The ophthalmoscope may further comprise a second filter disposed relative to the first filter to receive light from the first filter and more specifically light from the first light source which is transmitted by the first filter and/or light from the second light source which is reflected by the first filter.

The second filter may filter light of a further colour, i.e. light of a colour different from the colour of light filtered by the first filter, and which corresponds to the colour of light emitted by a third light source, for example amber light. The second filter may be dichroic.

In contrast with the first filter, the optical configuration of the ophthalmoscope may be such that the second filter may filter the light of the further colour by transmission instead of reflection. More specifically, the second filter may be transmissive of light of the further colour received from the first filter to thereby filter the light of the further colour. Otherwise the second filter may reflect the remaining light received from the first filter for onward transmission from the ophthalmoscope, such as through a window of the ophthalmoscope, for illumination of the eye. Alternatively or in addition, the first filter may be a bandpass filter, light of the further colour being transmitted because it falls in the passband of the shortpass filter.

Light received from the first filter may impinge on a first side of the second filter whereby light of the further colour comprised in the light received from the first filter is filtered, and more specifically is transmitted, by the second filter whereas light otherwise is reflected by the second filter. More specifically, the second filter may be oblique to, such as at 45 degrees to, the path of the light received from the first filter.

Light emitted by the third light source may impinge on a second, opposite side of the second filter. The light from the third light source may be transmitted by the second filter whereupon the transmitted light is combined with the light received from the first filter and reflected by the second filter to thereby in effect reintroduce the light transmitted and hence filtered by the second filter. The second filter may be oblique to, such as at 45 degrees to, the path of the light received from the third light source.

As mentioned above, the first to third light sources may be selectively operated to change the spectral composition of the light from the filter arrangement and more specifically the spectral composition of the light from the first and second filters. By way of example, and where the second light source emits red light and the third light source emits amber light, when the second and third light sources are inoperative, the first and second filters filter red and amber light from the white light received from the first light source whereby the light from the filter arrangement comprises more predominantly green/blue light.

The above-described arrangement of first to third light sources and first and second filters may be modified to include fourth and further light sources emitting light of yet further different spectral composition and third and further filters. For example, a fourth light source may emit blue light. Each of all but the last filter may be operative on light received from the first light source or preceding filter and from the respective light source in the same fashion as described above with respect to the first filter and second light source. Furthermore, the last filter may be operative on light received from the penultimate filter and from the last light source in the same fashion as described above with respect to the second filter and third light source.

References above to transmission and reflection of light may not be absolute in respect of extent of transmission or reflection, i.e. may not involve complete transmission or reflection, but may involve transmission and reflection of at least 70% and more specifically of at least 80%.

The ophthalmoscope may further comprise a collimating lens in front of the light source. Where the ophthalmoscope comprises a filter, the collimating lens may be in an optical path between the light source and the filter. The collimating lens may be a double-convex lens.

The plural light sources, for example the first to third light sources, may be at substantially the same distance from their respective filters.

The present ophthalmoscope may be configured for differential imaging in respect of single images and/or video. Where the ophthalmoscope is capable of performing differential imaging in respect of single images this may be in addition to the capability to acquire video as described above.

In a first form, differential imaging may be provided by acquiring at least one image when the eye is illuminated with light of different wavelength compositions, such as red light and amber light, either simultaneously or at different times and in the latter circumstance normally in quick succession. According to the first form, the ophthalmoscope may comprise plural light sources with each light source emitting light of a different spectral composition, as described above. The ophthalmoscope may be configured to operate in accordance with the first form. More specifically, the processor may be configured to control operation of the plural light sources and the imaging apparatus and with appropriate timing of control to perform image acquisition when there is at least one of: illumination with light of different wavelength compositions simultaneously, for example red light and amber light simultaneously; and illumination with light of different wavelength compositions at different times, for example red light and amber light at different times. Where the ophthalmoscope is capable of both modes of operation, i.e. simultaneously and at different times, the ophthalmoscope may be configured for selection between these modes of operation, such as by way of a user operable control comprised in the ophthalmoscope. Differential imaging according to the first form may provide for enhanced diagnosis on account of illumination with light of different wavelength compositions making certain pathologies more evident than would be evident from illumination with light of one wavelength composition alone. Alternatively, differential imaging according to the first form may provide for enhanced diagnosis on account of the ability to compare images acquired when there is illumination with light of different wavelength compositions at different times. Differences and/or similarities between such images may make certain pathologies more evident than would be evident from inspection of images acquired with light of one wavelength composition alone.

In a second form, differential imaging may be provided by acquiring at least two images at different times, and more specifically in quick succession, with illumination of the same spectral composition, for example white light or amber light. Differential imaging according to the second form may provide for enhanced diagnosis by revealing pathologies that change over time.

Light from the back of the eye is, in theory, collimated. The present ophthalmoscope may therefore merely need to bring light into focus onto the imaging apparatus. If the subject eye were optically perfect the ophthalmoscope would require no adjustment. However, most eyes have a refractive error. In light of this appreciation, the present inventors have devised an approach to addressing refractive error of the eye.

The approach to addressing refractive error may comprise the imaging apparatus of the ophthalmoscope being moved along the optical axis. The imaging apparatus may therefore be mounted in the ophthalmoscope for movement back and forward within the ophthalmoscope and more specifically for movement relative to the main body where the ophthalmoscope comprises the main body described above. Where the ophthalmoscope comprises the imaging lens arrangement described below, the imaging apparatus may be mounted for movement such that distance between the imaging apparatus and the imaging lens arrangement increases when moved back along the optical axis and decreases when moved forward along the optical axis.

The present approach differs from many focusing mechanisms which involve movement of lenses rather than the imaging apparatus. This is because movement of lenses is normally more quickly or accurately done than movement of imaging apparatus. The present approach therefore goes against the grain of the skilled person who is seeking quick and accurate focusing for the present ophthalmoscope.

When the refractive error of the eye has been addressed by focusing according to the present approach, there may be no need to perform focusing again because the eye aberration that gives rise to the refractive error does not normally change over time. Control of the ophthalmoscope to perform focusing, such as by way of the processor as described further below, may therefore take place only once for a particular eye and furthermore may take place during preparation for imaging, such as after alignment of the ophthalmoscope by way of the red light as described above.

The ophthalmoscope may comprise a focusing mechanism which provides for movement of the imaging apparatus back and forward along the optical axis. The focusing mechanism may comprise a motor, and more specifically a stepper motor, and a translating mechanism for translating rotation of the motor to linear movement. An input to the translating mechanism may be mechanically coupled to the motor and an output from the translating mechanism may be mechanically coupled to the imaging apparatus.

The translating mechanism may comprise a leadscrew. The motor may be mechanically coupled to the leadscrew whereby the motor rotates the leadscrew. The imaging apparatus may be mechanically coupled to the leadscrew by threaded engagement whereby the imaging apparatus moves in substantially the same direction as a longitudinal axis of the leadscrew.

As described above, the focusing mechanism addresses lack of focus caused by refractive error of a subject's eye by movement of the imaging apparatus along the optical axis. The ophthalmoscope may be configured to determine an extent of movement of the imaging apparatus along the optical axis to improve focusing. The ophthalmoscope may therefore comprise a driving algorithm which runs on the processor and which determines the extent of movement needed.

An out-of-focus image has lower level of detail than an in-focus image. The driving algorithm when run may therefore determine a level of detail in at least one image. The level of detail may be determined by computing an evaluation function and more specifically a frequency domain evaluation function for an image. Of frequency domain evaluation functions, the discrete cosine transform (DCT) may be preferred under certain circumstances. The driving algorithm when run may apply the DCT to an image acquired by the ophthalmoscope. More specifically, at least one cosine with the highest lambda may be identified in each of plural different parts of each transformed image and contributions from the plural different parts added to determine an overall level of detail for the image.

The level of detail in one image may be insufficient to determine whether or not the image is sufficiently in focus. Plural images may therefore be acquired by the ophthalmoscope each for a respective different position of the imaging apparatus along the optical axis. Furthermore, each of the plural images may be evaluated as to level of detail whereby the image of the plural images with the highest level of detail may be identified. In view of this, the driving algorithm may: control the focusing mechanism to move the imaging apparatus to each of plural different positions along the optical axis; control the imaging apparatus to acquire an image at each of the plural different positions; and evaluate each acquired image as to level of detail, such as by way of application of the DCT. The driving algorithm may further: identify the image of the plural acquired images having the most detail therein, such as in dependence on application of the DCT to each acquired image; and control the focusing mechanism to move the imaging apparatus to the position along the optical axis corresponding to the identified image. A peak in a curve formed in dependence on the frequency domain evaluation function transformed acquired plural images may correspond to the acquired image of greatest detail.

According to a first approach, the driving algorithm may control the focusing mechanism to move the imaging apparatus over a wide range of movement, such as over substantially the full range of movement, and may control the imaging apparatus to acquire a first plurality of images, each of the first plurality of images acquired at a respective one of a first plurality of steps taken during the wide range of movement. The driving algorithm may then fit a parabola to the levels of detail determined for the first plurality of images, determine a peak for the parabola, and then determine a first position for the imaging apparatus corresponding to the determined peak. Thereafter, the driving algorithm may control the focusing mechanism to move the imaging apparatus over a narrow range of movement which comprises the determined first position, may control the imaging apparatus to acquire a second plurality of images, each of the second plurality of images acquired at a respective one of a second plurality of steps taken during the narrow range of movement. The driving algorithm may then fit a parabola to the levels of detail determined for the second plurality of images, determine a peak for the parabola, and then determine a second position for the imaging apparatus corresponding to the determined peak. The second position for the imaging apparatus may be the position at which refractive error is addressed. This first approach may involve a small number of steps of movement of the imaging apparatus during acquisition of each of the first and second pluralities of images while compensating for refractive error to sufficient extent.

According to a second approach, and where the prescription for the eye is known, the driving algorithm may control the focusing mechanism to move the imaging apparatus to a first position along the optical axis corresponding to the prescription and to move the focusing mechanism one step forward and one step backward from the first position or two steps backward from the first position to second and third positions respectively. The driving algorithm may control the imaging apparatus to acquire an image at each of the first to third positions and to fit a parabola to the levels of detail for the three acquired images. The driving algorithm may further determine the peak of the parabola with the peak of the parabola corresponding to the image of greatest detail.

As described above, the present ophthalmoscope may comprise at least one light source and an imaging apparatus. Light from the at least one light source may be emitted in a first direction from the ophthalmoscope, such as through a window comprised in a main body of the ophthalmoscope, to the eye. An imaging path for the ophthalmoscope, i.e. a path from the illuminated eye to the ophthalmoscope and then within the ophthalmoscope to the imaging apparatus, may be in a second direction opposite to the first direction between the eye and the ophthalmoscope and then for part of the imaging path within the ophthalmoscope. The imaging apparatus may be disposed to receive images at an angle and more specifically at angle of between 60 and 90 degrees, and more specifically between 75 and 85 degrees, to a direction of propagation of light from the ophthalmoscope. There may therefore be need to redirect the imaging path from the second direction to a third direction whereby the image is oriented with the imaging apparatus whereby the image may be acquired properly by the imaging apparatus. Accordingly, the ophthalmoscope may further comprise a path redirecting arrangement which receives the image and redirects the image towards the imaging apparatus.

The path redirecting arrangement may comprise a first mirror which is disposed obliquely to the second direction to thereby receive the image from the eye and reflect the image in the third direction towards the imaging apparatus.

The path redirecting arrangement may further comprise a second mirror which is on the same side of the first mirror as the imaging apparatus. The second mirror may be disposed relative to the first mirror such that the second mirror receives light emitted along a light path by the at least one light source and reflects the received light onto the first mirror whereby the first mirror reflects the received light in the first direction towards the eye. The second mirror may be disposed relative to the first mirror such that the second mirror does not obscure the imaging path between the first mirror and the imaging apparatus.

The ophthalmoscope may further comprise a field lens arrangement which lies in the imaging and light paths and before light leaves the ophthalmoscope for the eye. The field lens arrangement may be between the first mirror described above and a widow comprised in the ophthalmoscope. The field lens arrangement may comprise a field lens pair and more specifically an achromatic lens pair. The field lens pair may have a focal length between 15 mm and 45 mm, 20 mm and 40 mm, or 25 mm and 35 mm. More specifically the field lens pair may have a focal length of 30 mm.

The ophthalmoscope may further comprise an imaging lens arrangement in the imaging path and disposed in front of the imaging apparatus. The imaging lens arrangement may comprise an imaging lens pair and more specifically an achromatic lens pair. The imaging lens pair may have at least one focal length between 5 mm and 35 mm, 10 mm and 30 mm, or 15 mm and 25 mm. More specifically the imaging lens pair may have focal lengths of 20 mm and 25 mm.

As described above, the ophthalmoscope is under the control of the processor such as in respect of selecting which of plural light sources are operative and operating the imaging lens arrangement to acquire video of a particular duration and perhaps also single images.

The processor may have the form of a microcontroller. The processor may be configured to perform one or more of the processes described herein. The ophthalmoscope may comprise structures and/or non-transitory memory having programmed instructions which are operated upon by the processor and perhaps further electronic circuitry to perform these processes.

The processor may comprise plural cores, such as four cores. A plural core architecture may be advantageous in view of simultaneous operations being carried out by the ophthalmoscope and furthermore in view of the processing intensive nature of certain operations and perhaps also a desire to complete processes quickly, such as focusing to address refractive error as described above.

The imaging apparatus may comprise an image sensor and more specifically a CMOS image sensor.

As described above, the ophthalmoscope may comprise a main body which supports and may also contain components of the ophthalmoscope. The main body may define a housing for the ophthalmoscope. The main body may comprise a handle portion, which may be gripped in one hand as described above, and a main body portion. A window through which the light and imaging paths pass may be comprised in the main body portion. The handle portion may extend from the main body portion. As described above, the light and imaging paths may pass in first and second directions through the window. A longitudinal axis of the handle portion may extend in a handle direction. The handle direction may be at an angle of between 100 and 120 degrees and more specifically between 105 and 115 degrees to the first and second directions. In a form suitable for certain applications, the handle direction may be at an angle of 110 degrees to the first and second directions. An ophthalmoscope of such form in respect of angulation of handle portion to main body portion may be more readily and comfortably used by affording ease of presentation of the window of the ophthalmoscope towards a patient's eye.

The main body may comprise a user operable control, e.g. one or more push button switches, to operate at least one of the light source and the imaging arrangement. Further to this, the ophthalmoscope may be subject to user control and/or configuration by way of a communication port. The communication port may be wired and/or wireless, such as Wi-Fi. More specifically, a user may determine control and/or configuration parameters by way of a computer, such as a smartphone or a personal computer of one kind or another, and may upload the control and/or configuration parameters to the ophthalmoscope by way of the communication port.

The ophthalmoscope may be configured for battery powered operation. The ophthalmoscope may therefore comprise a battery holder arrangement configured to hold at least one battery, and more specifically at least one rechargeable battery, and to provide for drawing of electrical power therefrom for operation of the light source and the imaging arrangement. Alternatively or in addition the ophthalmoscope may be configured to receive electrical power from an external source of power, such as by way of a wired link between the ophthalmoscope and other apparatus, such as a personal computer. Where the ophthalmoscope comprises at least one rechargeable battery, the ophthalmoscope may be configured for recharging of the at least one rechargeable battery. Recharging may be by electrical contact or may be contactless, such as in accordance with the Qi standard.

The ophthalmoscope may be configured for wireless and more specifically Wi-Fi transmission of data from the ophthalmoscope. Data such as acquired image data may be transmitted under control of the processor from the ophthalmoscope to remote computing apparatus, for example for analysis and/or storage. The ophthalmoscope may comprise data storage wherein acquired image data is buffered before onward transmission from the ophthalmoscope.

According to a second aspect of the present invention there is provided a method of acquiring video of an eye with an ophthalmoscope, the ophthalmoscope comprising a light source, an imaging apparatus, and a processor, the method comprising:

positioning the ophthalmoscope whereby a beam of non-coherent light emitted by the light source impinges on the eye and the imaging apparatus acquires plural images of at least part of the eye, each of the plural images acquired when non-coherent light from the light source impinges on the eye; and operating the processor to at least one of: control the imaging apparatus such that time adjacent images of the plural images are acquired with a predetermined period therebetween; and record a time of acquisition of each of a plurality of the plural images and to associate each time of acquisition with the respective image, whereby the acquired plural images are related in time to one another and thereby constitute video of the eye.

Embodiments of the second aspect of the present invention may comprise one or more features of the first aspect of the present invention.

The present inventors have appreciated the use of light of different wavelength compositions to be of wider applicability than hitherto described. Therefore, and according to a third aspect of the present invention, there is provided an ophthalmoscope configured to acquire at least one image of an eye, the ophthalmoscope comprising:
plural light sources each emitting a beam of non-coherent light of a different wavelength composition which in use impinges on the eye;
an imaging apparatus which acquires at least one image of at least part of the eye, the image acquired when non-coherent light from at least one of the light sources impinges on the eye; and
a processor configured to control the plural light sources and the imaging apparatus to acquire the at least one image of at least part of the eye.

The utility of light of different wavelength compositions is discussed above. The light of different wavelength compositions may impinge upon the eye at the same time when an image is acquired or may impinge upon the eye at different times, and more specifically at adjacent times, with an image acquired when each of the light of different wavelength compositions impinges upon the eye. The processor may be configured accordingly. More specifically, the processor may control the plural light sources to emit beams of light of different wavelength compositions at the same time and may control the imaging apparatus to acquire an image when the plural emitted beams of light of different wavelength compositions are impinging on the eye. Alternatively or in addition, the processor may control the plural light sources to emit beams of light of different wavelength compositions at different times and may control the imaging apparatus to acquire an image when each of the plural emitted beams of light of different wavelength compositions is impinging on the eye.

The ophthalmoscope may be configured as described above to provide for light of a first number of different wavelength compositions impinging on the eye when the ophthalmoscope has a second number of light sources, the first number being greater than the second number. More specifically, the ophthalmoscope may be optically configured to at least one of: combine light emitted by at least two light sources; and filter light emitted by at least one light source to thereby change the wavelength composition of the light emitted by the at least one light source. Further features in this regard are described above.

The ophthalmoscope may be configured as described above to acquire at least one of: a single image; plural images which are time unrelated to one another such that they do not constitute video; and plural images which are time related to one another such that they constitute video.

Further embodiments of the third aspect of the present invention may comprise one or more features of the first aspect of the present invention.

According to a fourth aspect of the present invention, there is provided a method of acquiring at least one image of an eye with an ophthalmoscope, the ophthalmoscope comprising plural light sources, an imaging apparatus, and a processor, the method comprising:
positioning the ophthalmoscope whereby a beam of non-coherent light emitted by at least one of the plural light sources impinges on the eye; and
operating the processor to control the plural light sources and the imaging apparatus to acquire at least one image of at least part of the eye when the beam of non-coherent light emitted by at least one of the plural light sources impinges on the eye.

Embodiments of the fourth aspect of the present invention may comprise one or more features of the first or third aspects of the present invention.

The present inventors have appreciated the use of non-coherent red light to be of wider applicability than hitherto described. Therefore, and according to a fifth aspect of the present invention, there is provided an ophthalmoscope configured to acquire at least one image of an eye, the ophthalmoscope comprising:
a light source emitting a beam of non-coherent red light which in use impinges on the eye;
an imaging apparatus which acquires at least one image of at least part of the eye when non-coherent red light from the light source impinges on the eye; and
a processor configured to control the imaging apparatus to acquire the at least one image of at least part of the eye.

As described above, the wavelength of the emitted red light may lie substantially in the range of 620 nm to 740 nm. Use of non-coherent red light may have the advantages discussed above. Advantage may be realised when a diagnostic image is acquired when the red light impinges on the eye.

Alternatively or in addition, the non-coherent red light may be used as a guide beam for alignment of the ophthalmoscope and before acquisition of a diagnostic image. The ophthalmoscope may be configured accordingly. More specifically, the ophthalmoscope may comprise a display which is viewable by a user of the ophthalmoscope. The ophthalmoscope may be configured, such as under control of the processor, to display on the display images acquired by the imaging apparatus when the non-coherent red light impinges on the eye. The emitted red light may therefore be used as a guide beam for alignment of the ophthalmoscope with the eye while feedback on alignment is provided to the user by way of the display.

Further embodiments of the fifth aspect of the present invention may comprise one or more features of the first aspect of the present invention.

According to a sixth aspect of the present invention, there is provided a method of acquiring at least one image of an eye with an ophthalmoscope, the ophthalmoscope comprising a light source, an imaging apparatus, and a processor, the method comprising:
positioning the ophthalmoscope whereby a beam of non-coherent red light emitted by the light source impinges on the eye; and
operating the processor to control the imaging apparatus to acquire at least one image of at least part of the eye when the beam of non-coherent red light impinges on the eye.

Embodiments of the sixth aspect of the present invention may comprise one or more features of the first aspect or fifth aspect of the present invention.

The present inventors have appreciated the above described approach to addressing refractive error of the eye to be of wider applicability than hitherto described. Therefore, and according to a seventh aspect of the present invention, there is provided an ophthalmoscope configured to acquire plural images of an eye, the ophthalmoscope comprising:
a light source emitting a beam of non-coherent light through a window of the ophthalmoscope whereby, in use, the beam of non-coherent light impinges on the eye;
an imaging apparatus which acquires plural images of at least part of the eye when non-coherent light from the light source impinges on the eye, the imaging apparatus mounted in the ophthalmoscope for movement back and forward within the ophthalmoscope to thereby change the length of an optical path between the imaging apparatus and the window; and
a processor configured: to control movement of the imaging apparatus within the ophthalmoscope; and to control the imaging apparatus to acquire each of the plural images at a respective different length of optical path between the imaging apparatus and the window.

The ophthalmoscope may be configured as described above to determine a length of optical path between the imaging apparatus and the window which addresses refractive error of the eye. Further embodiments of the seventh aspect of the present invention may comprise one or more features of any previous aspect.

According to an eighth aspect of the present invention there is provided a method of acquiring plural images of an eye with an ophthalmoscope, the ophthalmoscope comprising a light source, an imaging apparatus, and a processor, the method comprising:
positioning the ophthalmoscope whereby a beam of non-coherent light emitted by the light source passes through a window of the ophthalmoscope to thereby impinge on the eye; and
operating the processor: to control back and forward movement of the imaging apparatus within the ophthalmoscope to thereby change the length of an optical path between the imaging apparatus and the window; and to control the imaging apparatus to acquire each of the plural images at a respective different length of optical path between the imaging apparatus and the window and when non-coherent light from the light source impinges on the eye.

Embodiments of the eighth aspect of the present invention may comprise one or more features of any previous aspect of the present invention.

### BRIEF DESCRIPTION OF DRAWINGS

Further features and advantages of the present invention will become apparent from the following specific description, which is given by way of example only and with reference to the accompanying drawings, in which:

FIG. 1 is a perspective view from one side, the rear and below of an ophthalmoscope according to an embodiment of the present invention;

FIG. 2 is a schematic representation of optical components of the embodiment of FIG. 1;

FIG. 3A shows distribution of refractive error in the human eye;

FIG. 3B is a plot of the relationship between focus and stepper motor position in the ophthalmoscope;

FIG. 3C shows plots of stepper motor steps along the x-axis against level of detail in acquired images;

FIG. 4A is a plot of pupil radius over time;

FIG. 4B shows plots of sensitivity of S, M and L cone types against wavelength;

FIG. 4C shows plots of absorbance of cones and rods against wavelength; and

FIG. 4D shows plots of receptor density for cones and rods against extent of eccentricity.

### DESCRIPTION OF EMBODIMENTS

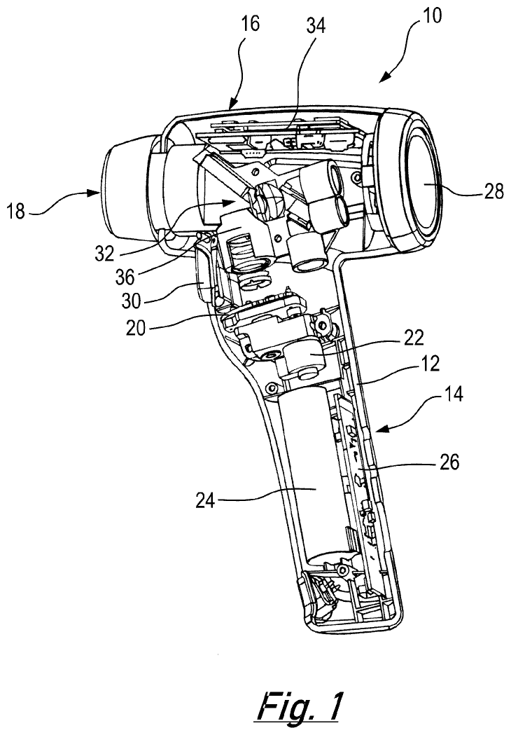

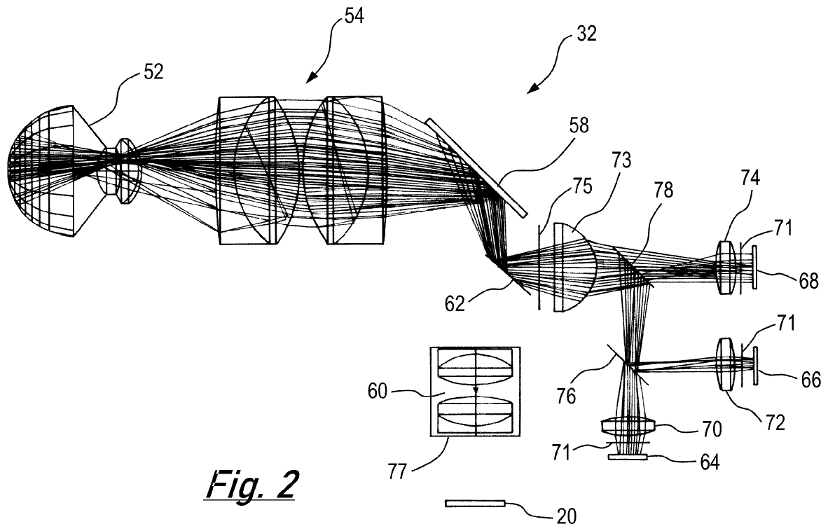

A perspective view of an ophthalmoscope 10 according to an embodiment of the present invention is shown in FIG. 1. The perspective view of FIG. 1 is from one side, the rear and below of the ophthalmoscope. Furthermore, one side of a housing 12 of the ophthalmoscope is removed to reveal the parts supported in the internal space defined by the housing. FIG. 2 is a schematic representation of optical components of the embodiment of FIG. 1. The ophthalmoscope 10 will now be described with reference to FIGS. 1 and 2.

The housing 12 (which constitutes a main body of the ophthalmoscope) comprises a handle portion 14, which is sized and shaped to be gripped in one hand by the clinician, and a main housing portion 16 (which constitutes a main body portion of the ophthalmoscope). A window 18 through which light and imaging paths of the ophthalmoscope pass is supported in one end of the main housing portion. The handle portion 14 and the main housing portion 16 are integrally formed from a right-hand half and a left hand half of the housing 12 whereby the handle portion extends from the underside of the main housing portion. The left and right halves of the housing 12 are formed from a plastics material such as ABS. The handle portion 14 is at an angle of 110 degrees to the underside of the main housing portion 16 extending away from where the handle portion joins the main housing portion towards the window. The angulation of the handle portion 14 provides for readiness and comfort of presentation of the window 18 of the ophthalmoscope towards a patient's eye.

The ophthalmoscope 10 comprises a CMOS image sensor 20 (which constitutes an imaging apparatus) which is mounted inside the handle portion 14. The CMOS image sensor 20 is an AR0820AT from On Semiconductor of 5005 East McDowell Road, Phoenix, AZ 85008 USA. The CMOS image sensor 20 is mounted inside the handle portion 14 for movement along a longitudinal axis of the handle portion whereby a distance between the CMOS image sensor 20 and the main housing portion 16 changes. Movement of the CMOS image sensor 20 is by way of a combined stepper motor and leadscrew 22 (which constitutes a focusing mechanism). The combined stepper motor and leadscrew 22 is a 15000 Series 15 mm (0.59-in) Can-Stack Stepper Motor Linear Actuator (External Linear with Ø ZBMR Nut version) from AMETEK. Inc. of 1100 Cassatt Road, Berwyn, PA 19312 USA. Electrical power for the stepper motor is provided by a rechargeable battery 24 mounted inside the handle portion 14. The rechargeable battery 24 is charged either by way of electrical contact or contactlessly in accordance with the Qi standard. Motor and battery control circuitry 26 for the stepper motor and the rechargeable battery 24 is comprised in a printed circuit board which extends alongside the rechargeable battery 24 inside the handle portion 14. Further circuitry to provide for operation of the CMOS image sensor 20 is comprised in a printed circuit board which supports the CMOS image sensor. The motor and battery control circuitry 26 and the further circuitry for the CMOS image sensor 20 is designed in accordance with data sheets for the combined stepper motor and leadscrew 22 and the CMOS image sensor 20 and otherwise in accordance with the ordinary design skills of the person skilled in the art.

The ophthalmoscope 10 further comprises a circular display 28 which is mounted on the opposite end of the main housing body 16 to the window 18. The display 28 is a PV13904PY24G-C1 1.39″ AMOLED display from Kingtech of 2^{nd} Floor, Building C, Jia Huang Yuan Technical Park, Tiegang, Xixiang, Bao'an District, Shenzhen, Guangdong, China 518126. The display 28 displays images acquired by the ophthalmoscope to the clinician. The ophthalmoscope also comprises a clinician operable trigger switch 30 which is located on the exterior of the handle portion 14 adjacent where the handle portion joins the main housing portion on the side of the handle portion closer to the window 18. The trigger switch 30 is thus disposed such that the clinician can grip the handle portion 14 and operate the trigger switch with one hand. The trigger switch 30 is depressed by the clinician to control operation of light beams emitted by the ophthalmoscope 10 and acquisition of images and video by the ophthalmoscope. The ophthalmoscope 10 comprises an optical assembly 32 and control and image processing circuitry 34 held inside the main housing body 16.

Components of the optical assembly 32 are mounted on a chassis 36 which is supported by the main housing body 16. The chassis 36 is formed from a plastics material such as ABS. The optical assembly 32 is described further below with reference to FIG. 2. The control and image processing circuitry 34 is comprised in a printed circuit board which extends alongside the optical assembly 32 on the side of the optical assembly opposite the side of the optical assembly facing the handle portion 14. The control and image processing circuitry 34 comprises a NXP i.MX 8M Mini™-based System-on-Module from Variscite of 4 Hamelacha Street, Lod, 7152008 Israel. The control and image processing circuitry 34: controls operation of the ophthalmoscope 10 in dependence on clinician operation of the trigger switch 30 and internally stored parameters; controls operation of the combined stepper motor and leadscrew 22 to provide focus for a particular patient; selectively controls operation of light sources comprised in the optical assembly 32 during use of the guide beam and during image and video acquisition; controls operation of the CMOS image sensor 20 to acquire images and video of the patient's eye; processes and stores acquired images and data derived therefrom; controls the display 28 to display acquired images and/or video; and provides for Wi-Fi communication with other computing apparatus, such as a Personal Computer (PC), by way of its Wi-Fi module. The Wi-Fi capability provides for onward transmission of acquired images and video and data derived therefrom, for downloading of firmware updates to the ophthalmoscope, and for downloading of configuration parameters to change the like of how light sources are selectively operated and characteristics of acquired images and video, such image resolution and length of video.

The optical assembly 32 will now be described with reference to FIG. 2. FIG. 2 represents an eye 52 of a patient in addition to the optical assembly 32. The eye 52 forms part of the optical system constituted by the ophthalmoscope 10 when it is in use in respect of the ophthalmoscope illuminating the eye and acquiring images and video of the illuminated eye. The optical assembly 32 comprises a field lens arrangement 54 which is supported inside the housing 12 near the window 18 to manipulate light beams emitted by the optical assembly 32 and passing through the window and images acquired by the CMOS image sensor 20 by way of the window. The field lens arrangement 54 comprises an objective lens pair having a focal length of 30 mm. The objective lens pair is a 30 mm achromatic pair 1:1 with 30 mm and 30 mm EFL achromats from Edmunds Optics Limited of Unit 1, Opus Avenue, Nether Poppleton, York, YO26 6BL, UK. Of the optical components of the optical assembly 32, the field lens arrangement 54 is disposed closest to the eye 52.

The optical assembly 32 also comprises a fold mirror 58 (which constitutes a first mirror) which is disposed on the image acquisition side of the field lens arrangement 54. The fold mirror 58 receives images from the eye by way of the field lens arrangement 54 and reflects the received images towards the CMOS image sensor 20. The fold mirror 58 also receives light emitted by the light sources of the optical assembly 32 and reflects the light towards the field lens arrangement 54. The optical assembly 32 yet further comprises an imaging lens arrangement 60 in the imaging path between the fold mirror 58 and the CMOS image sensor 20. The imaging lens arrangement 60 is constituted by a 15 mm achromatic pair 1:1.25 with 20 mm and 25 mm EFL achromats from Edmunds Optics Limited. The optical assembly 32 is configured such that the working distance between the field end of the ophthalmoscope 10 and the eye 52 being imaged is between 10 mm and 20 mm.

The optical assembly 32 further comprises a coupling mirror 62 (which constitutes a second mirror) which is disposed such that it is spaced apart from and partially overlapping with the fold mirror 58 whereby the coupling mirror does not obscure the imaging path between the fold mirror 58 and the CMOS image sensor 20. The coupling mirror 62 is angled to receive light emitted by the light sources of the optical assembly 32 and to redirect the received light towards the fold mirror 58 for onward reflection to the field lens arrangement 54.

The optical assembly 32 further comprises a white light source 64, a red light source 66, an amber light source 68, first to third collimating lenses 70, 72, 74, a first optical filter 76, and a second optical filter 78. Each of the first to third collimating lenses 70, 72, 74 is a 9 mm dia.×9 mm FL, VIS 0° Inked, Double-Convex Lens from Edmunds Optics Limited (part no. 47-485). The white light source 64 is a white light emitting LED, the red light source 66 is an LED emitting red light at 660 nm, and the amber light source 68 is an LED emitting amber light at 589 nm. The white light emitting LED 64 is a OSWC5111P 5 mm Warm White LED 28000mcd from TruOpto. The red light emitting LED 66 is a 2.5 V Red LED 5 mm Through Hole from Ledtech (UK) Ltd., Candela House, Cardrew Industrial Estate, Redruth, Cornwall, TR15 1SS, United Kingdom (part no. LURR5000H1D1). The amber light emitting LED 68 is an OVL-5526 Through Hole, T-1¾ (5 mm), 30 mA, 2.1 V LED from Premier Farnell Limited, 150 Armley Road, Leeds, LS12 2QQ, United Kingdom.

The white light source 64 emits light along a linear main light path between the white light source and the second optical filter 78. Each of the red and amber light sources 66, 68 emits light along a respective one of red and amber light paths which extend linearly and parallel to each other towards the main light path and on the same side of the main light path. The first collimating lens 70 is supported in front of the white light source 64 and in the main light path. The second collimating lens 72 is supported in front of the red light source 66 and in the red light path. The third collimating lens 74 is supported in front of the amber light source 68 and in the amber light path. The first optical filter 76 is a 730FDS12-KO dichroic shortpass filter from Knight Optical of Roebuck Business Park, Harrietsham, Kent, ME17 1AB, United Kingdom. The second optical filter 78 is a 640FDC12 dichroic bandpass filter also from Knight Optical. A diffuser 71 is located between each of the white, red and amber light sources 64, 66, 68 and its respective collimating lens 70, 72, 74. Each diffuser removes the fine structure of the respective LED from the illumination pattern. Each of the diffusers 71 is a 15° Diffusing Angle 8″×8″ Unmounted Sheet from Edmunds Optics Limited of Unit 1, Opus Avenue, Nether Poppleton, York, YO26 6BL, UK.

The first filter 76 is at 45 degrees to the main light path from the white light source 64. White light emitted by the white light source 64 impinges on a first side of the first filter 76 whereby light of the same and longer wavelength than the red light emitted by the red light source 66 is reflected and thereby filtered by the first filter whereas light otherwise is transmitted by the first filter. Red light emitted by the red light source 66 impinges on a second, opposite side of the first filter 76. The light from the second light source is reflected by the first filter 76 into the main light path whereby the reflected light from the second light source is combined with the light emitted from the white light source 64 and transmitted by the first filter.

The second filter 78 is at 45 degrees to the main light path from the white light source 64. Light received from the first filter 76 impinges on a first side of the second filter 78 whereby amber light corresponding to the amber light emitted by the amber light source 68 is transmitted and thereby filtered by the second filter whereas light otherwise is reflected by the second filter towards the coupling mirror 62. Amber light emitted by the amber light source 68 impinges on a second, opposite side of the second filter 78. The light from the amber light source 68 is transmitted by the second filter 78 whereby the transmitted light from the second filter is combined with the light received from the first filter and reflected by the second filter to thereby reintroduce the amber light filtered by the second filter.

The second filter 78 is disposed relative to the coupling mirror 62 such that light received from the first filter 76 and reflected by the second filter, and light from the amber light source 68 transmitted by the second filter is directed towards the coupling mirror 62. The light received by the coupling mirror 62 is reflected towards the fold mirror 58 for onward transmission to the field lens arrangement 54. A condenser lens 73 is present between the second filter 78 and the coupling mirror 62 to improve upon illumination efficiency. The condenser lens 73 is a 15 mm dia×12 mm FL, MgF2 Coated, Molded Aspheric Condenser Lens from Edmunds Optics Limited of Unit 1, Opus Avenue, Nether Poppleton, York, YO26 6BL, UK. A first rotatable linear polariser 75 is present between the condenser lens 73 and the coupling mirror 62. A second rotatable linear polariser 77 is present between the CMOS image sensor 20 and the imaging lens arrangement 60. The first and second linear polarisers 75, 77 work together to control reflex from the cornea of the eye 52 and from the field lens arrangement 54.

The white light source 64, the red light source 66, and the amber light source 68 are selectively operated under control of the control and image processing circuitry 34 to change the spectral composition of light falling on the coupling mirror 62 and hence the spectral composition of light illuminating the eye 52. By way of example, when the red light source 66 and the amber light source 68 are inoperative, the first and second filters 76, 78 filter red and amber light from the white light received from the white light source 64 whereby light falling on the coupling mirror 62 comprises predominantly green/blue light. By way of further example, when the white, red and amber light sources are all operative, the ophthalmoscope emits white light. In alternative embodiments, the presently described arrangement of three light sources and first and second filters is modified to include fourth and further light sources emitting light of yet further different spectral composition and third and further filters. For example, a fourth light source emits blue light. Each of all but the last filter is operative on light received from the white light source 64 or the preceding filter and from the respective light source in the same fashion as described above with respect to the first filter and the red light source 66. Furthermore, the last filter in the filter arrangement is operative on light received from the penultimate filter and from the last light source in the same fashion as described above with respect to the second filter and the amber light source 68.

Operation of the ophthalmoscope 10 will now be described. Before diagnostic images and video are acquired, the ophthalmoscope 10 is aligned in relation to the subject eye 52 by the clinician moving the ophthalmoscope. During alignment, the ophthalmoscope is operated to emit red light from the red light source 66 with the other light sources being inoperative whereby red light is emitted through the window 18. The red light is used by the clinician as a guide beam to aid alignment. During alignment, the ophthalmoscope is further operative to acquire a series of images by the CMOS image sensor 20 with the acquired images displayed on the display 28 to provide feedback to the clinician on positioning of the ophthalmoscope. Basis for use of red light in preference to infrared light is discussed below.

When alignment is complete, the ophthalmoscope 10 is operative to compensate for refractive error of the eye, should refractive error be present. According to a first approach to compensation, the CMOS image sensor 20 is driven by the combined stepper motor and leadscrew 22 under control of the of the control and image processing circuitry 34 to a starting position either at its closest position to the fold mirror 58 or at its furthest position from the fold mirror. During a first search stage, the CMOS image sensor 20 is driven stepwise from its starting position to the other of the closest position to the fold mirror and the furthest position from the fold mirror and an image is acquired by the CMOS image sensor at each of a small number of steps. The control and image processing circuitry 34 runs a driving algorithm, which processes the acquired images as follows further to controlling movement of the CMOS image sensor 20 and acquisition of images. The driving algorithm applies a discrete cosine transform (DCT) to each acquired image, identifies at least one cosine with the highest lambda in each of plural different parts of the acquired image, and sums contributions from the plural different parts to determine an overall level of detail for the acquired image. The driving algorithm then determines which of the acquired images represents the best focus location of the plural images acquired over the small number of steps. Thereafter the driving algorithm moves the CMOS image sensor 20 to carry out a second search stage. During the second search stage, the CMOS image sensor 20 is driven stepwise over a narrow range containing the best location determined by the first stage and an image is acquired by the CMOS image sensor at each of a small number of steps. The driving algorithm is then operative as per the first search stage to determine which of the images acquired during the second search stage represents the best focus location and causes the control and image processing circuitry 34 to control the stepper motor and leadscrew 22 to move the CMOS image sensor to this focus location. The driving algorithm thus adjusts focus of the ophthalmoscope 10 to compensate for the refractive error. When the refractive error of the eye has been addressed in this way, there is no need to repeat the present process again because the eye aberration that gives rise to the refractive error does not normally change over time.

According to a second approach to compensation, and where the prescription for the eye is known, the driving algorithm causes the control and image processing circuitry 34 to control the stepper motor and leadscrew 22 to move the CMOS image sensor 20 to a first position along the optical axis corresponding to the prescription and to move the CMOS image sensor one step forward and one step backward to second and third positions respectively. An image is acquired at each of the first to third positions. As described above with reference to the first approach to compensation, the driving algorithm applies the DCT to each of the three acquired images and determines a level of detail for each of the three acquired images from the DCT transformations. The driving algorithm then fits a parabola to the curve constituted by the levels of detail for the three acquired images and finds the peak of the parabola. The driving algorithm concludes its process by determining the location of CMOS image sensor 20 corresponding to the found peak and causes the control and image processing circuitry 34 to control the stepper motor and leadscrew 22 to move the CMOS image sensor to the determined location.

The direction of the ophthalmoscope 10 is liable to change during video acquisition because of the hand-held nature of the ophthalmoscope. The ophthalmoscope 10 therefore carries out digital image stabilisation. Image stabilisation comprises determining a rigid registration, which involves translation and rotation, between acquired images. The rigid registration is determined by determining an x, y translation between two acquired images by applying Fast Fourier Transform (FFT) matching and Normalised Cross Correlation (NCC) over a 512 by 512 central window in the images. The x, y translation is found by FFT deconvolution and finding a peak in the inverse shifted FFT result.

Now that alignment of the ophthalmoscope 10 and compensation for refractive error, if needed, are complete, operation progresses to acquisition of images and video of the patient's eye. The ophthalmoscope 10 provides for flexibility in respect of acquisition of images and video, such as the spectral composition of light used for illumination when images and video are acquired, the order of use of light of different spectral compositions, the length of video acquired, and frequency of acquisition of images for video. A rate of acquisition between 15 and 25 frames per second has been found to be advantageous in certain applications. How such flexibility is used depends on the clinical condition being monitored or screened for, on a patient's specific requirements, on the clinician's preferred approach to eye inspection, etc. According to a first example, the ophthalmoscope 10 is configured by way of firmware to acquire a single image when there is illumination with white light, then to acquire video when there is illumination with red light, and finally to acquire video when there is illumination with amber light. By way of a second example, and where the ophthalmoscope 10 comprises a fourth blue light emitting LED, the ophthalmoscope 10 is configured by way of firmware running on the control and image processing circuitry 34 to acquire a single image when there is illumination with white light, and then to acquire video when there is illumination with blue light. By way of a third example, acquisition of diagnostic images is as per one of the first and second examples and also to perform differential imaging. Differential imaging involves one, other or both of: illumination with lights of different spectral composition at the same time when an image is acquired; illumination with lights of different spectral composition at adjacent times and acquisition of an image at each of the adjacent times.

Video is acquired by way of the control and image processing circuitry 34 relating acquired plural images in time to one another. This is done by one, other or a combination of both of two approaches. According to the first approach, neighbouring images are acquired with a predetermined time between them. According to the second approach, acquired images are time stamped upon acquisition. The first and second approaches may be combined by, for example, time stamping first and second acquired images and then acquiring the third and further images with a predetermined time between acquisition of neighbouring images from the second image onwards.

Single images and video are displayed to the clinician on the display 28. Inspection of images and video on the display 28 may suffice for the clinician under certain circumstances. Further to display, the control and image processing circuitry 34 stores acquired images and video in a datastore comprised therein. The stored images and video are uploaded to the like of a Personal Computer by way of Wi-Fi communication for subsequent analysis and, if needed, to take advantage of review of images and video on a larger display.

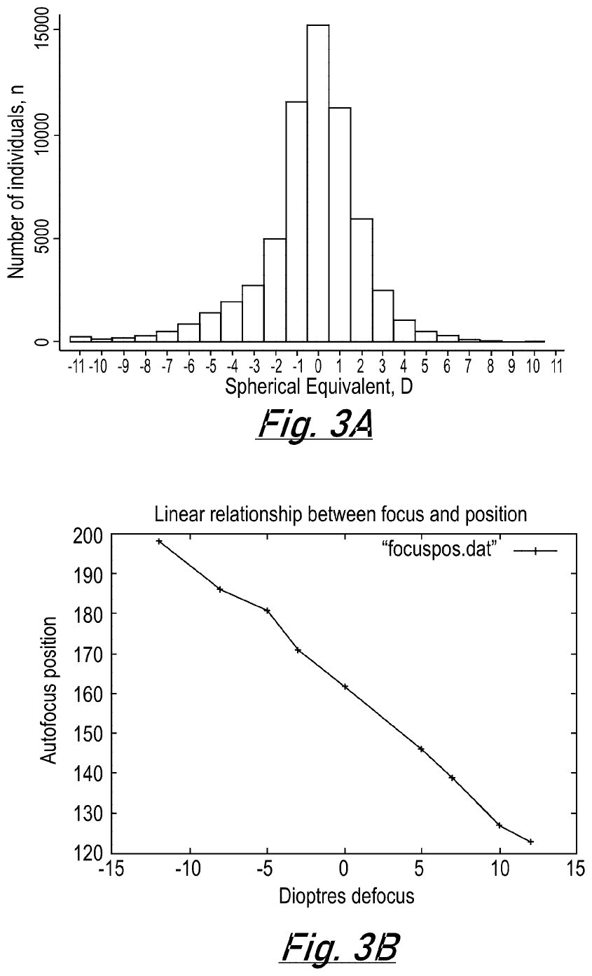

As described above, refractive error of the eye is compensated for by way of the driving algorithm. Most eyes have some refractive error. Distribution of refractive error in humans is shown in FIG. 3A. FIG. 3A is a bar chart of spherical equivalent in dioptres, D, along the x-axis and number of individuals, n, on the y-axis. FIG. 3B is a plot of the relationship between focus and stepper motor position of the ophthalmoscope 10 described above with dioptres defocus along the x-axis and stepper motor position in respect of number of steps along the y-axis. As can be seen from FIG. 3B, there is a linear relationship between focus and stepper motor position. FIG. 3C shows plots of stepper motor steps along the x-axis against level of detail in acquired images as determined by application of the DCT, as described above. Each plot is for a different starting extent of defocus between −12 and +12 dioptres in one dioptre steps. The letter ‘m’ in the legend of FIG. 3C means +D and the letter ‘p’ in the legend means −D with the following two numbers indicating the starting dioptre defocus, e.g. ‘00’ means 0 dioptres defocus and ‘05’ means 5 dioptres defocus. As can be seen from FIG. 3C, the ophthalmoscope 10 exhibits substantially unimodal behaviour, it being noted that the human brain is unlikely to be able to detect a defocus of 0.25 D when the focusing process of the ophthalmoscope is complete.

As described above, the ophthalmoscope 10 uses red light as a guide beam. This is in contrast to known ophthalmoscopes which use infrared light as a guide beam. Red light of the above-described wavelength and of a certain intensity was selected so as not to cause the pupil to reflexively contract but yet is of sufficient intensity to permit alignment and also diagnostic imaging.

Increasing stimulus intensity is associated with an increase in pupil response amplitude and maximum rate of pupil constriction and, it appears, pupil re-dilatation.

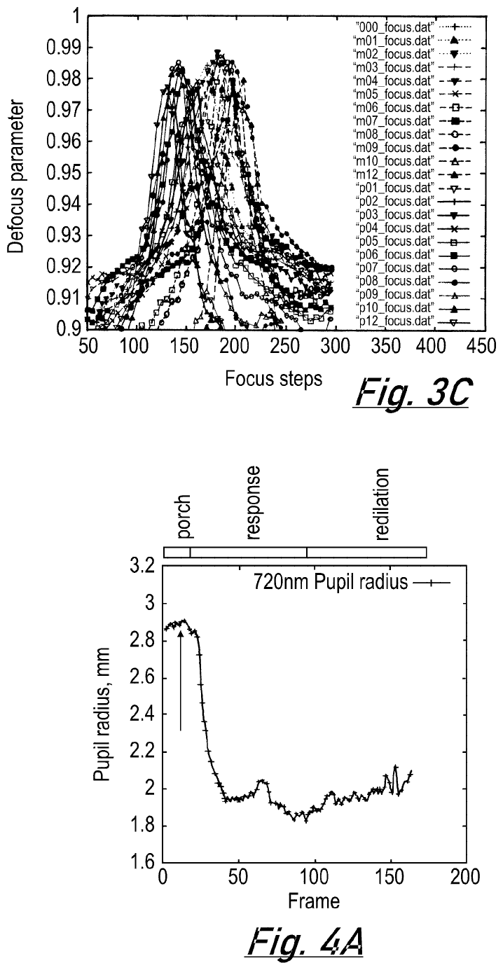

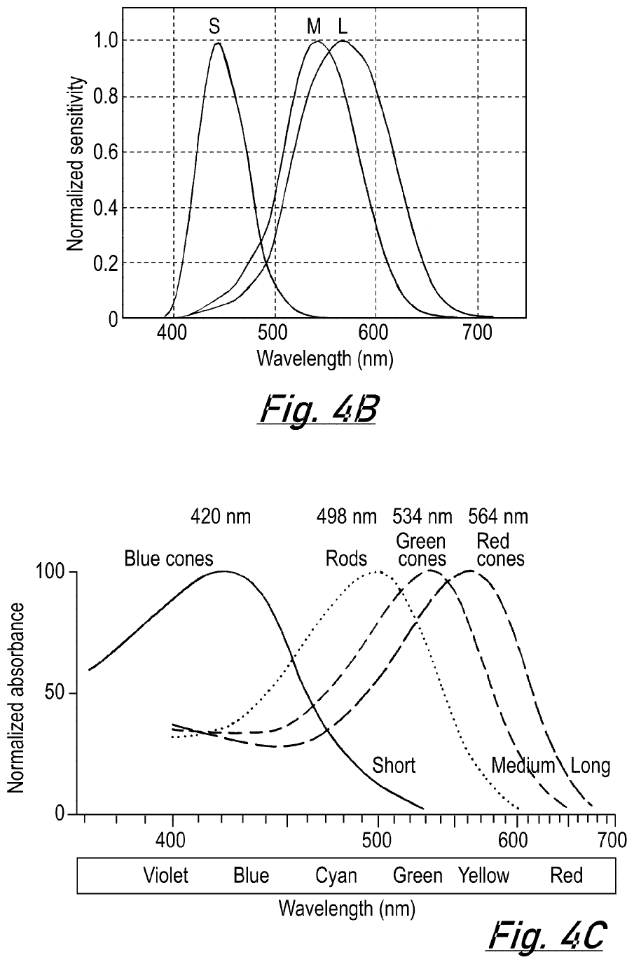

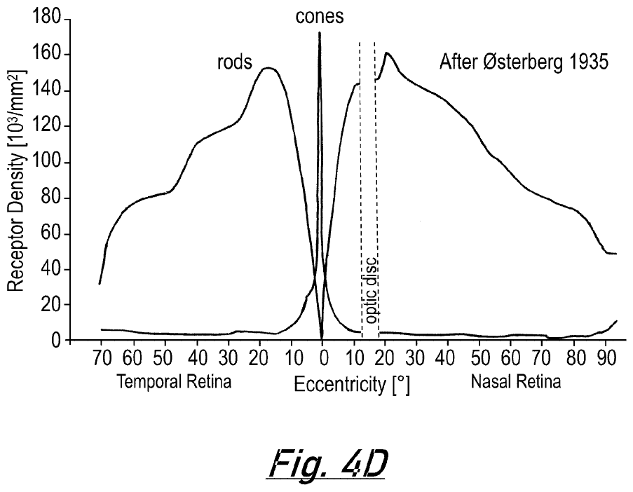

Latency from stimulus to onset of pupil response decreases with increasing stimulus intensity. Pupillary constriction occurs after a period of latency after stimulation (i.e. the porch). After constriction, pupillary re-dilation occurs, and the pre-stimulus resting diameter is reached. The rate of pupillary constriction increases rapidly to a maximum and then decreases progressively until the re-dilatation phase ensues. Similarly re-dilatation velocity increases to a maximum and then progressively decreases as the resting diameter is approached. This pattern of behaviour has been observed when illumination is maintained. In experiments performed in development of the present ophthalmoscope, the bright light illuminant was not switched off so experimental results represent what happens with the eye under constant stimulation. In the graph shown in FIG. 4A, the point of stimulus is shown by the arrow and the “frames” are measured at more-or-less one frame every 80 ms (12.5 fps). It was seen from the experiments that the typical response latency is around 400 to 1200 ms. It was noted that the majority of the latency in normal subjects is due to delay in iris smooth muscle contraction and that a relatively small part is due to conduction along the pupillary reflex pathway.

Turning away now from bright light, in the human eye cone cells are not functional after spending some time in low visible light. Cone cells are the main driver of pupillary constriction but when complete dark adaptation occurs (after around 20 minutes in the dark) the cone cells are essentially switched off. They may be reactivated very quickly though.

Normal vision uses the three cone types known as S, M and L (short, medium, and long wavelengths) and their firing wavelengths are shown in FIG. 4B. The graphs in FIG. 4B are gross simplifications of the range of human variation, however, both in their shape and dominant wavelength. The area of particular interest in development of the present ophthalmoscope is that over about 600 nm because that is where daylight vision starts to fail. Patients are likely to have a mix of firing S, M and L cones as well as firing rods hence their vision will involve a mix of these with the rods becoming active as the cones are starting to shut down. It is very unlikely that patients will be fully dark adapted during ophthalmoscopy, although they will be on the way to that. Scotopic vision is produced exclusively through rod cells, which are most sensitive to wavelengths of around 498 nm (green-blue) and are insensitive to wavelengths longer than about 640 nm (reddish orange). This condition is often called the Purkinje effect and means that red is not perceived in dim light. FIG. 4C shows that post-scotopia any wavelength from 600 nm upwards has very little effect on the pupil per se as it is almost impossible to be seen. Since the intended ophthalmology patients are dark adapting, rather than dark adapted, there will still be firing in the L cone cells but this will reduce over time. This means the final pupil constriction effect is a sum of L cone cell response and a small fraction of rod cell activity.

Scotopic vision occurs at luminance levels of 10⁻³ to 10⁻⁶ cd/m². Maintaining complete scotopia involves imaging with at most this amount of light. This is very unlikely as even the higher of these values is around the level of incidental light coming into an optometrists examination lane through and around blinds and under doors, etc. Mesopic vision occurs in intermediate lighting conditions (at a luminance level of 10⁻³ to 10^{0.5} cd/m²) and is effectively a combination of scotopic and photopic vision. In this area one needs to use a combination of wavelength plus intensity. That is to say, one needs to combine a wavelength that is on the borders of perception and at an intensity that minimizes contraction. In normal light (luminance level 10 to 108 cd/m²), the vision of cone cells dominates and is photopic vision. There is little prospect of imaging in these circumstances without a radical change to ophthalmoscope optics or a radical change in the expectations of the clinician.

Each of the conditions above are effectively weighted by the numbers of the two types of receptor cells around the eye orbit as shown in FIG. 4D.

The periphery of the eye has a complete lack of discriminatory ability with respect to high light conditions (cones) but the rods occupy a much wider area. This means that applying light to the periphery only in the dark is not a potential approach. Assuming use of a guide beam to adjust focus in a patient who is only partially dark adapted one should avoid the macula. This can be done with reference to FIG. 4D by approaching the eye at between 10 and 20 degrees off axis.

The above considerations point to wavelengths between 590 nm and around 720 to 740 nm (the approximate start of the infra-red spectrum) as appropriate for use in the present ophthalmoscope. Experiments were carried out on the human eye in this wavelength range. The experiments showed the following.
1) The amount of pupillary contraction is relatively very small under light in the deep red part of the spectrum.
2) Every experimental graph showed that the pupil undergoes re-dilation. From most of the experiments, re-dilation takes place in 1.6 to 4.0 seconds and the degree of re-dilation is up to 80% of the original size of the pupil at dark adaptation.
3) Re-dilation is better at the lower end of the spectrum (635 to 660 nm) than the higher end.
4) Experimental results were more variable at the lowest end, in the red/amber range.
5) Given that one can image successfully at a pupil radius of 3.5 to 4 mm, a wavelength in the range of 635 to 660 nm was found to be advantageous as a guide beam for the present ophthalmoscope.

1. An ophthalmoscope configured to acquire video of an eye, the ophthalmoscope comprising:
a light source emitting a beam of non-coherent light which in use impinges on the eye;
an imaging apparatus which acquires plural images of at least part of the eye, each of the plural images acquired when non-coherent light from the light source impinges on the eye; and
a processor configured to at least one of: control the imaging apparatus such that time adjacent images are acquired with a predetermined period therebetween; and record a time of acquisition of each of a plurality of the plural images and to associate each time of acquisition with the respective image, whereby the acquired plural images are related in time to one another and thereby constitute video of the eye.

2. The ophthalmoscope according to claim 1 configured to carry out digital image stabilisation of the acquired plural images.

3. The ophthalmoscope according to claim 1 shaped and sized to be gripped in one hand.

4. The ophthalmoscope according to claim 1, comprising a main body, the main body defining at least one internal space in which the light source, the imaging arrangement, and the processor are accommodated, the main body further defining a window through which the non-coherent light from the light source is directed towards the eye, and the imaging arrangement acquiring the plural images by way of an imaging path which passes through the window.

5. The ophthalmoscope according to claim 1, in which the processor controls the rate of acquisition of images by controlling the imaging apparatus, the plural images acquired at a rate of between 10 and 50 frames per second.

6. The ophthalmoscope according to claim 1 preceding claims configured to acquire lossless video.

7. The ophthalmoscope according to claim 1, any one of the further comprising a display which is viewable by a user of the ophthalmoscope, the light source emitting a beam of non-coherent red light, plural images acquired by the imaging apparatus displayed on the display when non-coherent red light from the light source is emitted from the ophthalmoscope, whereby the emitted red light is used as a guide beam for alignment of the ophthalmoscope with the eye while feedback on alignment is provided to the user by way of the display.

8. The ophthalmoscope according to claim 7, in which the wavelength of the emitted red light lies in the range of 620 nm to 700 nm.

9. The ophthalmoscope according to claim 1, in which the light source emits light of different wavelength compositions at different times, and video of the eye is constituted for the light of each of the different wavelength compositions.

10. The ophthalmoscope according to claim 9, in which the light source emits a first beam of non-coherent amber light and a second beam of substantially white light, the first and second beams emitted at different times, and video of the eye is constituted for emission of each of the first and second beams.

11. The ophthalmoscope according to claim 9, which is optically configured to at least one of: combine light emitted by at least two light sources when the ophthalmoscope comprises plural light sources each emitting light of a respective different wavelength composition; and filter light emitted by at least one light source, to thereby change the wavelength composition of light emitted from the ophthalmoscope and which in use impinges on the eye.

12. The ophthalmoscope according to claim 1, which is configured for differential imaging in respect of at least one of a single acquired images and video constituted by the plural acquired images.

13. The ophthalmoscope according to claim 12, in which differential imaging comprises acquiring at least one image when the eye is illuminated with light of different wavelength compositions.

14. The ophthalmoscope according to claim 12, in which differential imaging comprises acquiring at least two images at different times with emitted light of substantially the same spectral composition.

15. The ophthalmoscope according to claim 1, in which the imaging apparatus is mounted in the ophthalmoscope for movement back and forward within the ophthalmoscope relative to at least one imaging lens comprised in the ophthalmoscope, said movement under control of the processor, to thereby provide for focusing to address refractive error of the eye.

16. The ophthalmoscope according to claim 15, further comprising a focusing mechanism which moves the imaging apparatus back and forward, the focusing mechanism comprising a stepper motor and a translating mechanism which translates rotation of the stepper motor to linear movement of the imaging apparatus.

17. The ophthalmoscope according to claim 1, further comprising an optical path redirecting arrangement which receives the plural images and redirects the plural images towards the imaging apparatus, light emitted from the light source emitted in a first direction within the ophthalmoscope, an imaging path for the plural acquired images within the ophthalmoscope extending in a second direction opposite to the first direction between the eye and the ophthalmoscope and then for part of the imaging path within the ophthalmoscope, the optical path redirecting arrangement changing the imaging path from the second direction to a third direction.

18. The ophthalmoscope according to claim 17, in which the path redirecting arrangement comprises a first mirror which is disposed obliquely to the second direction to thereby receive the plural images and to reflect the plural images in the third direction towards the imaging apparatus.

19. The ophthalmoscope according to claim 18, in which the path redirecting arrangement further comprises a second mirror which is on the same side of the first mirror as the imaging apparatus, the second mirror disposed relative to the first mirror such that the second mirror receives light emitted by the light source and reflects the received light onto the first mirror whereby the first mirror reflects the received light in the first direction towards the eye, the second mirror disposed relative to the first mirror such that the second mirror does not obscure the imaging path between the first mirror and the imaging apparatus.

20. A method of acquiring video of an eye with an ophthalmoscope, the ophthalmoscope comprising a light source, an imaging apparatus, and a processor, the method comprising:
positioning the ophthalmoscope whereby a beam of non-coherent light emitted by the light source impinges on the eye and the imaging apparatus acquires plural images of at least part of the eye, each of the plural images acquired when non-coherent light from the light source impinges on the eye; and
operating the processor to at least one of: control the imaging apparatus such that time adjacent images of the plural images are acquired with a predetermined period therebetween; and record a time of acquisition of each of a plurality of the plural images and to associate each time of acquisition with the respective image, whereby the acquired plural images are related in time to one another and thereby constitute video of the eye.
